(19) United States
(12) Patent Application Publication
(10) Pub. No.: US 2021/0015883 A1
(43) Pub. Date: Jan. 21, 2021

(54) A NOVEL SYNERGIC HERBAL FORMULATION FOR THE PREVENTION AND TREATMENT OF PRE-DIABETES, DIABETES AND OTHER INSULIN RESISTANCE CASES

(71) Applicant: Lal HINGORANI, Petlad (IL)

(72) Inventor: Lal HINGORANI, Petlad (IL)

(21) Appl. No.: 17/043,923

(22) PCT Filed: Jul. 3, 2019
(86) PCT No.: PCT/IB2019/055685

§ 371 Date: Sep. 30, 2020

(30) Foreign Application Priority Data
Jul. 13, 2018 (IN) 201721025106

(51) Int. Cl. A61K 36/9066; A61K 36/185; A61K 31/685; A61K 31/12; A61K 31/375; A61K 31/192; A61K 36/48; A61K 36/28; A61K 33/24; A61P 3/10

(52) U.S. Cl. CPC A61K 36/9066; A61K 36/185; A61K 31/685; A61K 31/12; A61P 3/10; A61K 31/192; A61K 36/48; A61K 36/28; A61K 33/24; A61K 31/375

(57) ABSTRACT

The present invention relates to a novel synergic herbal formulation comprising Curcuma longa and Phylanthus emblica in precise combination along with one or more other ingredient for prevention and treatment of pre-diabetes, diabetes mellitus and other insulin resistance cases. Further the present invention provides a process for preparing the novel synergistic herbal formulation.

### FIELD OF INVENTION

This invention relates to a novel synergic herbal formulation. The present invention also relates to a process for preparing the novel synergistic herbal formulation. The present invention also relates to method of prevention and treatment of pre-diabetes, diabetes mellitus and other insulin resistance cases using novel synergistic herbal formulation.

### BACKGROUND INFORMATION

Diabetes is a chronic condition with high level of glucose in blood. To lower this increase level of glucose, β-cells of pancreas produces insulin. Absence or insufficient insulin production by pancreas leads to the condition called as Diabetes mellitus.

According to the s 2016 data from the (WHO), globally, an estimated 422 million adults are living with Diabetes mellitus. Diabetes prevalence is increasing rapidly; previous 2013 estimates from the International Diabetes Federation put the number at 381 million people having diabetes. In India diabetes currently affects more than 62 million people, which is more than 7.1% of the adult population. The average age on onset is 42.5 years. Nearly 1 million Indians die due to diabetes every year.

People with diabetes don't know that they have diabetes until they undergo blood sugar test. Over the period of time if untreated diabetes leads to blindness, nerve damage, kidney failure. Diabetes is also a leading cause of coronary heart disease and strokes due to its adverse effect of small vessels. Diabetes accelerates hardening and narrowing of arteries which leads to coronary heart disease. Medical cost to the people with diabetes is double than those do not have diabetes.

With diabetes mellitus, either human body doesn't make enough insulin or it can't use the insulin it does produce, or a combination of both.

There are two types of diabetes, type-1 (insulin dependent) and type-2 (non-insulin dependent). Type-1 diabetes is an autoimmune disorder, where pancreas is damaged and does not produce insulin.

Whereas in type-2 diabetes which is common and milder form of diabetes, β-cells of pancreas usually produces some insulin, but either the amount produced is not enough for the body's needs, or the body's cells are resistant to it. Insulin resistance, or lack of sensitivity to insulin, happens primarily in fat, liver, and muscle cells.

Insulin is a hormone that allows body cells to utilize sugar. Due to the developed resistance to insulin cells cannot utilize glucose and the blood glucose level goes high. Due to this increased glucose level pancreas starts to produce more insulin.

The beta cells in the pancreas try to keep up with this increased demand for insulin by producing more. As long as the beta cells are able to produce enough insulin to overcome the insulin resistance, blood glucose levels stay in the healthy range.

Over the period of time beta cells fail to keep up with the body's increased demand for insulin and this increased insulin resistance causes excess glucose levels in the bloodstream, leading to diabetes, pre-diabetes, and poly cystic ovarian disorder.

Pre-diabetes is a condition in which blood glucose or A1C levels, which reflect average blood glucose levels, are higher than normal but not high enough for a diagnosis of diabetes.

Pre-diabetes usually occurs in people who already have insulin resistance. In pre-diabetes, the beta cells can no longer produce enough insulin to overcome insulin resistance, causing blood glucose levels to rise above the normal range.

Once a person has pre-diabetes, continued loss of beta cell function usually leads to type 2-diabetes. People with type 2 diabetes have high blood glucose. Studies have shown that most people with pre-diabetes develop type 2 diabetes within 10 years, unless they change their lifestyle.

Some herbs which have their origin in Ayurveda have been tried to assess their efficacy in insulin resistance cases such as pre-diabetes and poly-cystic ovarian disorder and are found to be effective in reducing insulin resistance as well as seen to be reducing glucose level in diabetes conditions.

Ayurveda has mentioned Diabetes under the heading of Prameha. There are two types of prameha, 1) Sahaj prameha and 2) Apathyanimittaj prameha. The apathyanimittaj prameha is resembles the pre-diabetic or early type-2 diabetic condition, if it is not managed on time, can lead to chronic type-2 diabetes.

In ayurvedic medicine, Vagbhata has advocated a combination of Haridra and Amalaki known as Herbal formulation for the treatment of Prameha. The dose of this formulation varies from 3-5 gm twice a day depending upon patient.

Due to the taste and the daily requirement of Herbal formulation patient compliance to this formulation is very low. So there was a need to invent a new formulation of haridra and amalaki which will reduce daily requirement of this formulation as well as mask the unpleasant taste of it.

Still there is a need in the society to have a formulation for the prevention and treatment of diabetes mellitus which is effective and also have palatable taste.

Inventors of present invention have surprisingly found that a unique formulation of these herbs with some excipients like lecithin and turmeric oil which have synergic effect for better efficacy, better palatability and because of synergism, dose also can be reduced.

### OBJECTIVE OF INVENTION

Primary objective of the invention is to provide a unique herbal formulation which will not only prevent but also help to control diabetes mellitus.

Another objective of this formulation is to use it in the insulin resistance cases like Pre-diabetes condition and Polycystic Ovarian Disorder.

### SUMMARY OF INVENTION

The main aspect of the present invention is to provide a novel synergistic herbal formulation.

As per one aspect of the present invention is to provide a novel synergistic herbal formulation comprising:
(a) 20% to 30% by w/w of Curcuma longa alcohol extract,
(b) 5% to 10% by w/w of Curcuma longa water extract,
(c) 1% to 10% by w/w of Turmerone oil,
(d) 20% to 50% by w/w of Phylanthus emblica juice powder,
(e) 20% to 50% by w/w of Phylanthus emblica extract,
(f) 10% to 15% by w/w of Phosphatidyl choline

Another aspect of the present invention is to provide a process for preparing the novel synergistic herbal formulation of present invention.

As per another aspect of the present invention is to provide a novel synergistic herbal formulation which reduces insulin resistance and blood glucose levels in diabetic patients.

As per another aspect of the present invention is to provide a novel synergistic herbal formulation which is useful in pre diabetic condition, diabetes mellitus, insulin resistant diabetes, Polycystic ovarian disorder (PCOS).

### DETAILED DESCRIPTION OF THE DRAWINGS

FIG. 1. Herbal formulation (HF) on PPAR gamma gene expression by Real Time PCR.

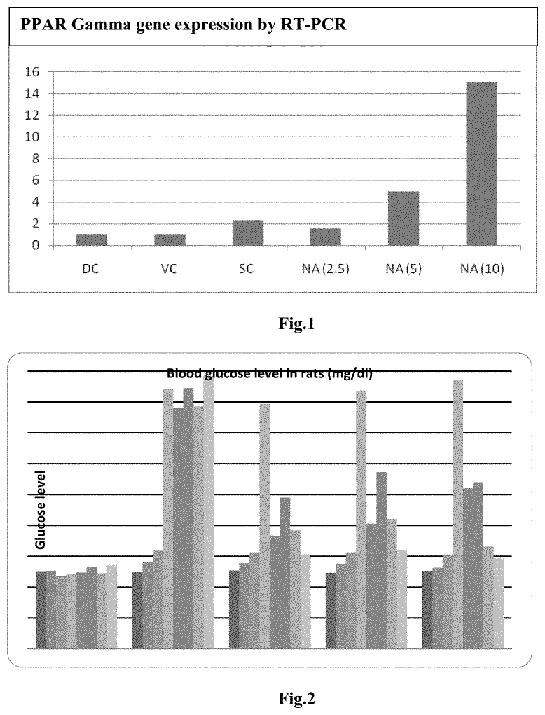

FIG. 2. Blood glucose levels in rats (mg/dL).

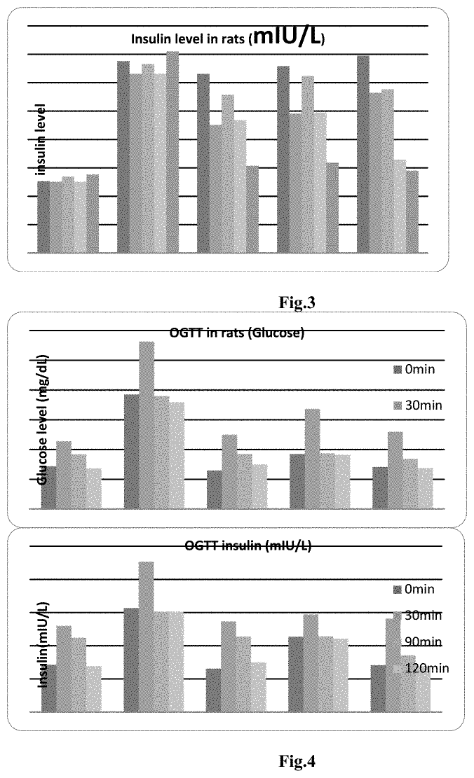

FIG. 3. Insulin levels in rats (mIU/L).

FIG. 4. Ogtt in rats in rats (Oral glucose tolerance test).

FIG. 5. Homa-ir and quicki in rats.

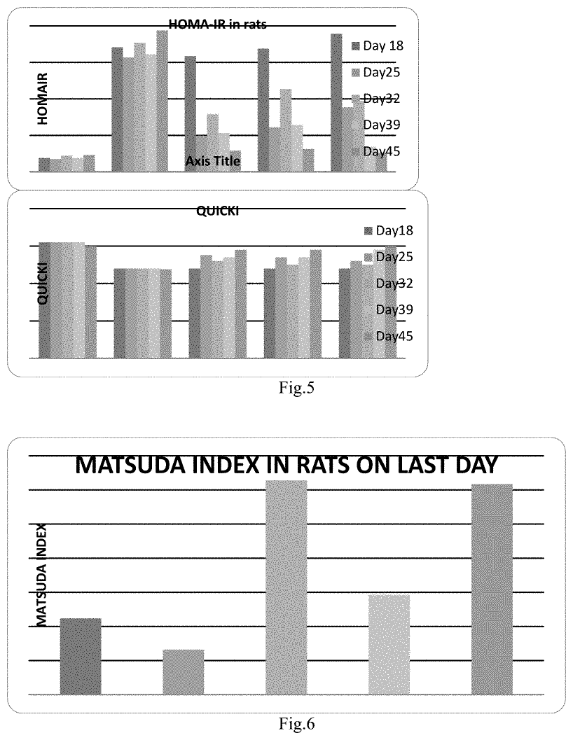

FIG. 6. Matsuda index in rats.

### DETAILED DESCRIPTION OF THE INVENTION

The present invention relates to a novel synergistic herbal formulation which is useful in reduces insulin resistance and blood glucose levels in diabetic patients.

The novel synergistic herbal formulation of present invention is effective in in pre diabetic condition, diabetes mellitus, insulin resistant diabetes, Polycystic ovarian disorder (PCOS).

The novel synergistic herbal formulation of present invention is prepared with different ingredients in different concentrations.

Herbal formulation comprises of Curcumin, Curcuma longa water extract, Turmerone oil Phylanthus emblica juice, Phylanthus emblica extract and the bioavailability enhancer Phosphatidyl choline. We have done two different studies to prove efficacy of this herbal formulation for the prevention and treatment of pre-diabetes, diabetes mellitus and other insulin resistance cases.

The invention involves the synergic formulation of herbs to reduce the insulin resistance which is a key pathology for development of pre-diabetes, diabetes, poly-cystic ovarian disorder and other similar disorders. The formulation also reduces the increased level of blood glucose in diabetes. The formulation is the combination of Curcuma longa alcohol extract, Curcuma longa water extract, Turmerone oil Phylanthus emblica juice, Phylanthus emblica extract and the bioavailability enhancer Phosphatidyl choline.

Having a dual mechanism of action of insulin sensitizing and anti-hyperglycemic the formulation is well introduced as for prevention and treatment of diabetes mellitus.

The present novel synergistic herbal formulation contains ingredients as listed below:

Curcuma longa Alcohol Extract and Curcuma longa Water Extract

Curcuma longa water and alcohol extract both are extracted from Curcuma Longa Rhizomes and is 90% to 95% Curcuminoids. Curcuma longa water extract contains 30% to 50% of polysaccharides and turmerone oil contains 50% to 70% α-turmerone.

Turmerone Oil

Turmerone oil is oil obtained from Curcuma longa rhizome.

Curcuma longa rhizomes were procured from Vedant trading, Shirad shahapur, Dist-Hingoli, Maharashtra

Phylanthus emblica Juice Powder

Phylanthus emblica juice powder contains 0.2 to 2% Vitamin C and 4 to 5.5% Gallic acid.

Phylanthus emblica Extract

Phylanthus emblica extract contains 30 to 45% of tannins and 10 to 12% Gallic acid.

Emblica officinalis fruits were procured from Amin Farm, Diguncha, Mokashan, Tal-Kalol, Dist Gandhinagar, Gujarat.

Phosphatidyl Choline

Phosphatidylcholines (PC) are a class of phospholipids that incorporate choline as a headgroup. They are a major component of biological membranes and can be easily obtained from a variety of readily available sources, such as egg yolk or soybeans, from which they are mechanically or chemically extracted using hexane. They are also a member of the lecithin group of yellow-brownish fatty substances occurring in animal and plant tissues. Dipalmitoyl phosphatidylcholine (a.k.a. lecithin) is a major component of pulmonary surfactant and is often used in the L/S ratio to calculate fetal lung maturity. While phosphatidylcholines are found in all plant and animal cells, they are absent in the membranes of most bacteria, including Escherichia coli. Purified phosphatidylcholine is produced commercially.

As per one embodiment, the Phosphatidyl choline is used in the range from 10 to 25% w/w, preferably in the range from 10 to 15% w/w.

As per one embodiment the novel herbal formulation of present invention may also contains other ingredients like diluent, binder, disintegrate, lubricant.

As per one embodiment, the novel herbal formulation of present invention may contain one insulin sensitizer.

As per one embodiment, the novel herbal formulation of present invention may contain chromium as insulin sensitizer in the range from 0.5 ppm to 5 ppm.

The novel synergistic formulation of present invention offers many advantages against the conventional herbal or ayurvedic formulations available for the prevention or treatment of pre diabetic condition, diabetes mellitus, insulin resistant diabetes, Polycystic ovarian disorder (PCOS).

| | Conventional | |
|---|---|---|
| | herbal/Ayurvedic | |
| | formulation | |
| | available | Novel herbal formulation |
| Dosage | 3-5 gms twice a day | 1 capsule twice a day |
| Taste | Slightly bitter | Tasteless as it is in |
| | | capsule form and |
| Convenience | Inconvenient to take | Convenient to consume |
| Composition | It is in crude form | This formulation contains |
| | and don't have any | specified ingredients in |
| | synergistic effect | specific ratio with other |
| | | ingredients which providing |
| | | synergistic effect. |
| Efficacy | Individual ingredients | Synergic effect is |
| | have weak antidiabetic | significantly higher than |
| | activity. | individual ingredient |

The effectiveness and synergistic activity of herbal formulation of present invention is evaluated by doing various studies where individual components are evaluated in comparison with final formulation as well as the final composition is evaluated in comparison with marketed formulation.

### EXAMPLE

### Example 1: Curcuma Longa Whole Extract

Curcuma longa whole extract was prepared by mixing Curcuma longa extract (NLT 90% curcuminoids), Curcuma longa water extract and Curcuma longa oil in the proportion mentioned in following table:

| S. No. | Ingredients details | % |
|---|---|---|
| 1 | Curcuma longa alcohol extract- | 85 |
| 2 | Curcuma longa water extract | 10 |
| 3 | Curcuma longa oil | 5 |

### Example 2: Phyllanthus Emblica Whole Extract

Phyllanthus emblica whole extract was prepare by mixing Emblica officinalis Extract and Emblica officinalis Juice powder in the proportion mentioned in following table:

| S. No. | Ingredients details | % |
|---|---|---|
| 1 | Emblica officinalis Extract | 66.67 |
| 2 | Emblica officinalis Juice powder | 33.33 |

### Example 3: Novel Herbal Formulation

| S. No. | Ingredients details | % |
|---|---|---|
| 1 | Curcuma longa alcohol extract | 28.1 |
| 2 | Curcuma longa water extract | 1.76 |
| 3 | Curcuma longa oil | 1.27 |
| 4 | Phosphatidylcholine | 12.65 |
| 5 | Emblica officinalis Extract | 40.41 |
| 6 | Emblica officinalis Juice powder | 15.81 |

Procedure:

Curcuma longa rhizome's alcoholic extract, its water extract and oil were transferred in extraction vessel containing alcohol and mixed properly in the proportion mentioned in table below. Phosphatidylcholine dissolved in alcohol and transferred to same vessel. Mixture was distilled at lower temperature to remove alcohol and complex formation. Paste was taken out from vessel and homogenized in homogenizer. Later Emblica officinalis fruit extract and Emblica officinalis fruit Juice powder were separately mixed in water at the proportion mentioned in table below. This solution mixture was added to the homogenizer containing Curcuma longa extract mixture with phosphatidylcholine. Complete solution mixture was homogenized for 3 hours and spray dried to get free flow powder.

### Example 4

Mixture was prepared by mixing Curcuma longa alcoholic extract and Phyllanthus emblica extract in the proportion mentioned in following table:

| S. No. | Ingredients details | % |
|---|---|---|
| 1 | Curcuma longa alcoholic extract | 33.33% |
| 2 | Phyllanthus emblica extract- | 66.67% |

### Example 5

Mixture was prepared by mixing Curcuma longa water extract and Phyllanthus emblica extract in the proportion mentioned in following table:

| S. No. | Ingredients details | % |
|---|---|---|
| 1 | Curcuma longa water extract | 33.33% |
| 2 | Phyllanthus emblica extract | 66.67% |

### Example 6

Mixture was prepared by mixing Berberis aristata extract and Phyllanthus emblica extract in the proportion mentioned in following table:

| S. No. | Ingredients details | % |
|---|---|---|
| 1 | Berberis aristata extract | 33.33% |
| 2 | Phyllanthus emblica extract | 66.67% |

### Example 7: Evaluation of Novel Herbal Formulation Through PPAR Gamma Expression Study

PPAR Gamma assay is a tool to analyze insulin sensitizing activity of any medicine. We have done PPAR Gamma assay of this Herbal formulation to find out its insulin sensitizing activity and it is compared with the available standard drug Pioglitazone.

Study Objective:

To evaluate the effect of the given samples on PPAR gamma expression.

Herbal Formulation for Study:

Samples of Herbal formulation were evaluated over a concentration range from 0.5 to 10 μg/ml. Pioglitazone, a known anti-diabetic agent was used as the standard control at a dose of 6 μg/ml; a dose extrapolated from the median therapeutic human dose. The Herbal formulation was dissolved in NAOH (0.1N).

Viability Studies:

Viability studies were carried out to eliminate cytotoxic concentrations of the samples using MTT assay. Based on the results of the MTT assay, the following concentrations were used for the PPAR Expression studies. Concentration selected was as follows 2.5, 5, 10 μg/ml.

Study Methodology:

Cell Culture and Adipocyte Differentiation:

3T3L1 Fibroblasts were grown in DMEM medium, supplemented with 10% FBS, in a humidified atmosphere of 5% CO2 at 370 C. On attaining 75-80% confluency, the cells at a concentration of 1×104 cells/ml, were seeded in 24 well plates. Differentiation was induced by supplementing the media with a cocktail of 1 mg/L insulin, 100 mg/L isobutyl-1-methylxanthine (IBMX) and 0.1 mg/L dexamethazone for 48 hrs followed by insulin alone for an additional 48 hrs. The media was then replaced with fresh culture medium (DMEM supplemented with 10% FBS) after 2 days and then every 2 day after upto 12 days. During differentiation samples were added to the cell culture medium at the concentration selected from the viability studies followed by addition of the samples after every 2 days along with the media replacement. Pioglitazone treated cells group was used as a standard control group where the untreated differentiated cells served as the Control group. For Herbal formulation which was soluble in 0.1N NaOH, the results were compared with another vehicle control group of 0.1 N NaOH treated cells.

Analysis of PPAR Gamma Expression:

On the 12th day after differentiation, total RNA was extracted with TRIzol reagent according to the manufacturer's instructions from the differentiated adipocytes. The concentration and purity of RNA were determined by measuring the absorbance at 260 nm (A260) and the ratio of the absorbance at 260 nm to the absorbance at 280 nm (A260/A280), respectively. Complementary DNA was synthesized with the RevertAid cDNA Synthesis Kit (Fermentas, Austin, Tex.) using 2.0 μg of total RNA. SYBR Green chemistry was used to perform the quantitative determination of the relative expression of PPAR gene. The PPAR gene was analyzed using the Rotogene Q; Qiagen real-time polymerase chain reaction machine. The primers used in the experiments are shown as follows:

Genes Primer Sequence Accession No.

GAPDH 5′-GTATGACTCCACTCACGGCAAA-3′
BC083080 5′-GGTCTCGCTCCTGGAAGATG-3′
PPAR 5′-TGTGGGGATAAAGCATCAGGC-3′
NM011146 5′-CCGGCAGTTAAGATCACACCTAT-3′

The reaction volume was 20 μl. All results were obtained from at least three independent experiments. Transcript levels were normalized to GAPDH (glyceraldehyde-3-phosphate dehydrogenase) levels. The relative messenger RNA (mRNA) expression levels were calculated according to the comparative CT (^{ΔΔ}CT) method. The target quantity is normalized to endogenous control i.e., Untreated Differentiated Cell Control group and Vehicle Control and is calculated using the formula: Target amount 2−^{ΔΔ}CT.

Results and Conclusion:

Herbal Formulation (HF) on PPAR Gamma Gene Expression by Real Time PCR

| Samples | PPARg CT | GAPDH CT | DCT | DDCt | 2\hat{ }-DDCT |
|---|---|---|---|---|---|
| DC | 28.12 | 15.09 | 13.03 | 0 | 1 |
| VC | 28.49 | 17.03 | 11.46 | 0 | 1 |
| SC | 25.69 | 15.426667 | 10.263 | −1.197 | 2.2920947 |
| HF- (2.5) | 26.93 | 15.31 | 11.62 | −0.64 | 1.55 |
| HF- (5) | 26.72 | 16.77 | 9.95 | −2.30 | 4.92 |
| HF- (10) | 24.26 | 15.92 | 8.34 | −3.91 | 15.01 |

Herbal formulation samples showed concentration dependent increase in PPAR gamma expression as compared to differentiated control (DC) and to 0.1 N NaOH treated cell control (VC). However maximum expression was seen in cells treated with Herbal formulation of Example 3 i.e., a 15-fold increase. As expected, Pioglitazone treated cells (SC) also showed a two fold increase in the PPAR gamma expression at the concentration studied.

### Example 8: Evaluation of Novel Herbal Formulation Through Animal Study

Evaluation of antidiabetic effect of herbal formulation against high fat diet and Streptozotocin (stz) induced insulin resistance in rats.

Test drug: Herbal formulation as per Example 4. It is used to control blood glucose levels in diabetic patients.

Treatment drug: Reference drug metformin (120 mg/kg, p.o)

Toxicant drug: High fat diet (HFD) and STZ (40 mg/kg, i.p)

Composition of High Fat Diet

| S. No. | Ingredients | Diet g/kg |
|---|---|---|
| 1 | Powdered normal pellet diet | 365 |
| 2 | Lard | 310 |
| 3 | Casein | 250 |
| 4 | Cholesterol | 10 |
| 5 | DL-methionine | 03 |
| 6 | Yeast powder | 01 |
| 7 | Sodium chloride | 01 |

Composition of Normal Pellet Diet

| S. No. | Ingredients | Diet g % |
|---|---|---|
| 1 | Crude protein | 25 |
| 2 | Crude oils | 11.04 |
| 3 | Crude fibres | 5.3 |
| 4 | Carbohydrates | 47.5 |
| 5 | Ash | 7.22 |
| 6 | Vitamins | 2.26 |
| 7 | Sand silica | 1.32 |

All values are expressed as mean±SEM; N=6 in each group.

One way ANOVA followed by Tukey-Kramer multiple comparison test is applied for statistical analysis *p<0.05, **p<0.01 and ***p<0.001 when experimental groups compared with HFD+STZ group.

Procedure:

Rats were divided 5 into groups of 8 rats each and treated in the following way:

Group I Normal control group—Received normal chow diet and drinking water daily for 45 days and citrate buffer (1 mL/kg, i.p) on the 15^{th} day of start of experiment.

Group II Toxicant Control: Received HFD throughout the experiment (45 days) and STZ (40 mg/kg, i.p) on the 15^{th} day of start of experiment.

Group III Herbal formulation I as per Example 4 (500 mg): Received HFD throughout the experiment (45 days), STZ (40 mg/kg, i.p) on the 15^{th} day of start of experiment and the Herbal formulation (500 mg/kg, p.o) from the 18th day for the next 28 days.

Group IV Herbal formulation II as per Example 4 (1000 mg): Received HFD throughout the experiment (45 days), STZ (40 mg/kg, i.p) on the 15^{th} day of start of experiment and the Herbal formulation (1000 mg/kg, p.o) from the 18th day for the next 28 days.

Group V Reference standard: Receive HFD throughout the experiment (45 days), STZ (40 mg/kg, i.p) on the 15th day of start of experiment and Metformin, (120 mg/kg) from the 18th day for the next 28 days.

The rats were allocated into dietary regimens by feeding HFD (58% fat, 25% protein and 17% carbohydrate, as a percentage of total kcal) and drinking water ad libitum, respectively, for the initial period of 2 weeks. After 2 weeks of dietary manipulation, the group of rats fed with HFD were injected a single low dose of STZ (40 mg/kg, i.p), while the Normal control rats were given the vehicle citrate buffer (pH 4.4) in a dose volume of 1 mL/kg, i.p. The fasting blood glucose (FBG) were measured 3 days after the STZ injection. The rats with FBG of more than 250 mg/dL were considered diabetic and selected for further pharmacological studies and their dosing with the Herbal formulations commenced and continued for the next 28 days. The rats were allowed to continue to feed on their respective diets until the end of the study. All FBG and insulin levels were determined on different days viz. 18^{th} day (3^{rd} day after STZ injection), 25^{th}, 32^{nd}, 39^{th} and 45^{th} days (24 h after the previous dose) by collecting blood from the tail vein/retro orbital plexus. On the 44^{th} day Oral Glucose Tolerance Test (OGTT) was performed wherein animals of all groups received glucose solution (1.5 g/kg) 30 minutes after the above treatments. Blood glucose and insulin levels were determined at 0, 30, 60, 90, 120 min after glucose administration. Blood glucose levels were measured using a digital glucometer. Insulin was assayed by using a RIA kit.

Insulin resistance is calculated using the following methods:

1. Homeostasis Model Assessment-Insulin Resistance

Homeostasis model assessment was first developed in 1985 by Matthews et al. It is a method used to quantify insulin resistance and beta-cell function from basal (fasting) glucose and insulin (or C-peptide) concentrations. HOMA is a model of the relationship of glucose and insulin dynamics that predicts fasting steady-state glucose and insulin concentrations for a wide range of possible combinations of insulin resistance and β-cell function. Insulin levels depend on the pancreatic β-cell response to glucose concentrations while, glucose concentrations are regulated by insulin-mediated glucose production via the liver. Thus, deficient β-cell function will echo a diminished response of β-cell to glucose-stimulated insulin secretion. Similarly, insulin resistance is reflected by the diminished suppressive effect of insulin on hepatic glucose production. The HOMA model has proved to be a robust clinical and epidemiological tool for the assessment of insulin resistance. HOMA describes this glucose-insulin homeostasis by means of a set of simple, mathematically-derived nonlinear equations. The approximating equation for insulin resistance has been simplified; it uses a fasting blood sample. It is derived from the use of the insulin-glucose product, divided by a constant.

The product of FPG×FPI is an index of hepatic insulin resistance.

The equation proposed by Matthews et al.:

IR_{HOMA}=I₀/(22.5×e^{-In(Go)} could be rewritten as:

IR_{HOMA}=(I₀×G₀)/22.5(mathematically:e^{-Inx}=1/x)

I₀—Fasting plasma insulin concentration (mIU/L),
G₀—Fasting plasma glucose concentration (mg/dL),

Blood Glucose Levels in Rats (Mg/dL)

| | Normal | | Metformin | | |
|---|---|---|---|---|---|
| Date | Control | HFD + STZ | 120 | HF-500 | HF-1000 |
| Day 1 | 124.71 ± 2.05 | 124.12 ± 4.12 | 126.87 ± 2.41 | 123.25 ± 4.62 | 126.00 ± 4.50 |
| Day 7 | 126.25 ± 2.78 | 140 ± 2.80* | 138.63 ± 1.54 | 138.00 ± 3.10 | 131.38 ± 3.51 |
| Day 15 | 117.87 ± 2.85 | 159 ± 4.10*** | 156.37 ± 3.17 | 156.13 ± 2.91 | 152.87 ± 5.93 |
| Day 18 | 120.83 ± 1.97 | 421.00 ± 56.53** | 396.67 ± 43.72 | 418.5 ± 49.04 | 436.5 ± 65.53 |
| Day 25 | 123.83 ± 3.32 | 391.00 ± 43.79*** | 183.167 ± 13.58*** | 202.83 ± 21.72*** | 260.33 ± 37.03* |
| Day 32 | 132.67 ± 4.73 | 422.67 ± 43.49*** | 245.167 ± 28.86** | 286.33 ± 25.42* | 269.83 ± 25.86** |
| Day 39 | 122.67 ± 3.73 | 392.83 ± 30.39*** | 191.83 ± 25.08*** | 210.17 ± 4.52*** | 165.67 ± 8.58*** |
| Day 45 | 135.33 ± 4.56 | 443.00 ± 12.75*** | 152.83 ± 8.40*** | 158.83 ± 8.63*** | 146.67 ± 5.51*** |

Insulin Levels in Rats (mIU/L)

| | Normal | | Metformin | | |
|---|---|---|---|---|---|
| Date | Control | HFD + STZ | 120 | HF-500 | HF-1000 |
| — | 12.65 ± 1.05 | 33.81 ± 4.89 *** | 31.56 ± 1.95 | 32.93 ± 3.26 | 34.74 ± 1.94 |
| Day 25 | 12.55 ± 0.61 | 31.56 ± 2.86*** | 22.56 ± 2.24* | 24.57 ± 1.39 | 28.20 ± 1.89 |
| Day 32 | 13.44 ± 0.93 | 33.3 ± 2.32** | 27.84 ± 5.014 | 31.17 ± 4.57 | 28.82 ± 1.37 |
| Day 39 | 12.54 ± 0.62 | 31.58 ± 5.52** | 23.40 ± 3.47 | 24.71 ± 1.18 | 16.41 ± 2.31* |
| Day 45 | 13.83 ± 0.69 | 35.52 ± 2.07*** | 15.36 ± 0.58*** | 15.91 ± 1.27*** | 14.5 ± 1.07*** |

OGTT IN RATS (Oral Glucose Tolerance Test)

| | | | Normal | | Metformin | | |
|---|---|---|---|---|---|---|---|
| TIME | | | Control | HFD + STZ | 120 | HF-500 | HF-1000 |
| 0 | Min | Glucose | 143.67 ± 1.96 | 385 ± 2.43*** | 129.5 ± 1.17*** | 185 ± 1.34*** | 141.5 ± 1.74*** |
| | | (mg/dL) | | | | | |
| | | Insulin | 14.25 ± 0.58 | 31.37 ± 1.63*** | 13.12 ± 0.97*** | 22.73 ± 1.14*** | 14.13 ± 0.93*** |
| | | (mIU/L) | | | | | |
| 30 | Min | Glucose | 227.67 ± 20.15 | 563.00 ± 7.04*** | 249.50 ± 0.56*** | 336.33 ± 4.31*** | 259.50 ± 5.10*** |
| | | (mg/dL) | | | | | |
| | | Insulin | 25.954 ± 0.68 | 45.33 ± 1.18*** | 27.308 ± 1.4*** | 29.37 ± 1.57*** | 28.15 ± 1.13*** |
| | | (mIU/L) | | | | | |
| 90 | Min | Glucose | 183.5 ± 2.23 | 379.83 ± 3.86*** | 185.167 ± 2.08*** | 187 ± 2.2*** | 168.17 ± 2.16*** |
| | | (mg/dL) | | | | | |
| | | Insulin | 22.43 ± 1.6 | 30.22 ± 1.46** | 22.72 ± 1.49** | 22.80 ± 0.19** | 17.13 ± 0.78*** |
| | | (mIU/L) | | | | | |
| 120 | Min | Glucose | 136.17± 2.91 | 358 ± 2.39*** | 149.83 ± 2.81*** | 182 ± 4.22*** | 137.33 ± 2.59*** |
| | | (mg/dL) | | | | | |
| | | Insulin | 13.85 ± 0.49 | 30.27 ± 0.86*** | 14.95 ± 1.035*** | 22.05 ± 1.31*** | 13.83 ± 1.12*** |
| | | (mIU/L) | | | | | |

2. Quantitative Insulin Sensitivity Check Index (QUICKI)

Quantitative insulin sensitivity check index (QUICKI) is an empirically-derived mathematical transformation of fasting blood glucose and plasma insulin concentrations that provide a consistent and precise ISI with a better positive predictive power. It is simply a variation of HOMA equations, as it transforms the data by taking both the logarithm and the reciprocal of the glucose-insulin product, thus slightly skewing the distribution of fasting insulin values. QUICKI has been seen to have a significantly better linear correlation with glucose clamp determinations of insulin sensitivity than minimal-model estimates, especially in obese and diabetic subjects. It employs the use of fasting values of insulin and glucose as in HOMA calculations. QUICKI is virtually identical to the simple equation form of the HOMA model in all aspects, except that a log transform of the insulin glucose product is employed to calculate QUICKI. The QUICKI can be determined from fasting plasma glucose (mg/dl) and insulin (μIU/ml) concentrations.

QUICKI=1/(log I₀+log G₀)

Results of Homa-Ir and QuickI in Rats

| | Index | Normal | | Metformin | | |
|---|---|---|---|---|---|---|
| DATE | type | Control | HFD + STZ | 120 | HF-500 | HF-1000 |
| Day 18 | HOMA-IR | 3.78 ± 0.32 | 34.16 ± 5.17*** | 31.7 ± 4.75 | 33.75 ± 4.06 | 37.86 ± 6.60 |
| | QUICKI | 0.31 ± 0.0033 | 0.24 ± 0.003*** | 0.24 ± 0.0057 | 0.24 ± 0.0033 | 0.24 ± 0.0044 |
| Day 25 | HOMA-IR | 3.5 ± 0.28 | 31.33 ± 5.62*** | 9.91 ± 0.69*** | 12.18 ± 1.2 *** | 17.7 ± 2.05* |
| | QUICKI | 0.31 ± 0.0034 | 0.24 ± 0.0054*** | 0.276 ± 0.0021*** | 0.27 ± 0.0033*** | 0.26 ± 0.0036*** |
| Day 32 | HOMA-IR | 4.41 ± 0.41 | 35.36 ± 5.08*** | 15.8 ± 2.76** | 22.66 ± 4.9 | 19.43 ± 2.46* |
| | QUICKI | 0.31 ± 0.0036 | 0.24 ± 0.0049*** | 0.26 ± 0.0042** | 0.25 ± 0.0055 | 0.25 ± 0.0033 |
| Day 39 | HOMA-IR | 3.78 ± 0.17 | 32.18 ± 8.49*** | 10.56 ± 1.7** | 12.76 ± 0.45* | 6.81 ± 1.12*** |
| | QUICKI | 0.31 ± 0.0022 | 0.24 ± 0.0061*** | 0.27 ± 0.0055*** | 0.27 ± 0.00** | 0.29 ± 0.0060*** |
| Day 45 | HOMA-IR | 4.63 ± 0.25 | 38.76 ± 2.20*** | 5.81 ± 0.39*** | 6.23 ± 0.60*** | 5.23 ± 0.38*** |
| | QUICKI | 0.30 ± 0.0033 | 0.238 ± 0.0016*** | 0.29 ± 0.0030*** | 0.29 ± 0.0042*** | 0.30 ± 0.0025*** |

HF-500: Herbal formulation as per Example 4 (500 mg)
HF-1000: Herbal formulation as per Example 4 (1000 mg)

3. Matsuda Index

Several methods have been described that derive an ISI from the OGTT. In these methods, the ratio of plasma glucose to insulin concentration during the OGTT is used. A novel assessment of insulin sensitivity that is simple to calculate and provides a reasonable approximation of whole-body insulin sensitivity from the OGTT was developed by Matsuda and Defronzo, and is referred to as the Matsuda index. Here the OGTT ISI (composite) was calculated using both the data of the entire 3 h OGTT and the first 2 h of the test. The composite whole-body insulin sensitivity index (WBISI), developed by Matsuda and DeFronzo is based on insulin values given in microunits per milliliter (μU/mL) and those of glucose, in milligrams per deciliter (mg/L) obtained from the OGTT and the corresponding fasting values The index of whole-body insulin sensitivity combines both hepatic and peripheral tissue insulin sensitivity. This index is calculated from plasma glucose (mg/dl) and insulin (mIU/l) concentrations in the fasting state and during OGTT.

$$\begin{matrix}Matsuda \\ index \end{matrix} 10 \text{,} 000 \text{/} \sqrt{\begin{matrix}( fasting G \times fasting I ) \\ ( mean G \times mean I ) \end{matrix}}$$

I₀—Fasting plasma insulin concentration (mIU/l),

G₀—Fasting plasma glucose concentration (mg/dl),

Gₘₑₐₙ—Mean plasma glucose concentration during OGTT (mg/dl),

Iₘₑₐₙ—Mean plasma insulin concentration during OGTT (mU/1),

10,000—Simplifying constant to get numbers from 0 to 12.

√—Correction of the nonlinear values distribution.
A. Animals used: 40
B. Species/Common name: Albino Wistar rats
C. Age/weight/size: Ten weeks, 150-180 g
D. Gender: Male
E. Number to be used (Year-wise breakups and total figures needed to be given): 40
F. Number of days each animal will be housed: 3 months
G. Details of injections schedule:
H. Details of withdrawal:
   Volumes: 2 mL per animal by retro-orbital plexus for insulin assay and 0.1 mL by tail vein for blood glucose.
I. Sites: Retro-orbital plexus, tail vein

| | | | | Volume/ | |
|---|---|---|---|---|---|
| Substances | Doses | | Sites | 100 g | |
| Herbal formulation of Example 4 | 500 | mg/kg | p.o | 1 | mL |
| Herbal formulation of Example 4 | 1000 | mg/kg | p.o | 2 | mL |
| Metformin | (120 | mg/kg), | p.o | 1 | mL |
| STZ | (40 | mg/kg) | i.p | 0.1 | mL |

MATSUDA INDEX IN RATS (A Matsuda value of less than 4.3 indicates insulin resistance.)

| | Normal | HFD + | Metformin | | |
|---|---|---|---|---|---|
| | Control | STZ | 120 | HF-500 | HF-1000 |
| Matsuda | 4.47 ± | 2.64 ± | 12.56 ± | 5.84 ± | 12.34 ± |
| Index | 0.15 | 0.05*** | 0.05*** | 0.08*** | 0.08*** |

HF-500: Herbal formulation as per Example 4 (500 mg)
HF-1000: Herbal formulation as per Example 4 (1000 mg)

Results and Conclusion

1. There was a significant increase in blood glucose levels (BGL) in all groups treated with HFD for 2 weeks when compared with Normal control animals. The increase in BGL in the 2^{nd} week of HFD feeding was significantly higher than in the first week.

2. After administration of STZ on the 15^{th} day of the study, after 3 days, i.e. on the 18^{th} day, BGL levels in all animals treated with STZ shot up tremendously when compared with the Normal Control animals.

3. There was a gradual decrease in BGL over 4 weeks of treatment with metformin or formulation of Example 4

4. At the end of the study, BGL of the HFD+STZ group was significantly elevated when compared with the Normal Control group, whereas BGL of Herbal formulation of Example 4 and metformin treatment groups were significantly lower when compared with the HFD+STZ group. The BGL levels of these groups were not significantly different from the Normal group. Though not significantly different from each other, Herbal formulation of example 4 (1000 mg) was better than metformin which was slightly better than Herbal formulation of example 4 (500 mg) treatment.

5. For insulin levels, the same trend as that for BGL was seen with minor differences in intermediate readings. At the end of the study while insulin levels remained significantly elevated in the HFD+STZ group of rats when compared with the Normal Control rats, the treatment groups exhibited significantly lowered insulin levels when compared with the HFD+STZ treatment and were comparable to the Normal Control insulin levels. Also, there was no significant difference between Herbal formulation of Example 4 (500 mg), Herbal formulation of Example 4 (1000 mg) and metformin treatments.

6. For OGTT, both, BGL and insulin levels were determined at various time points. The HFD+STZ group was always significantly higher than Normal Control for both BGL and insulin assay. However, throughout the study, all the three treatment groups exhibited significantly lower BGL and insulin levels when compared with the HFD+STZ group. At the end of the study, while all treatment groups could significantly display lower BGL and insulin levels, Herbal formulation of Example 4 (1000 mg) exhibited near normal BGL and insulin levels, followed by metformin and Herbal formulation of Example 4 (500 mg) treatment.

7. Following is a comparison of the three methods used in the study for determining insulin resistance:

In our experiment, in the HOMA IR method, insulin resistance was found to be significantly elevated in the HFD+STZ group of animals when compared with Normal Control animals, indicating insulin resistance. All treatment groups showed insulin resistance comparable with HFD+STZ group at the start of the experiment after the STZ dose. In the following weeks, insulin resistance was found to have been significantly attenuated by the treatment groups. At the end of the study all 3 treatment groups were comparable in reducing insulin resistance significantly and showed levels comparable to Normal group. Herbal formulation of Example 4 (1000 mg) was the nearest to Normal, followed by metformin and Herbal formulation of Example 4 (500 mg) treatments.

QUICKI values range between 0.45 in healthy individuals (noted as unusually healthy in the original study) and 0.30 in diabetics. Lower values reflect greater resistance with values below 0.339 indicating insulin resistance.

For the QUICKI measure, HFD+STZ treatment elicited a significantly lower value when compared with Normal Control value. All treatment groups elicited values significantly lower than Normal and similar to HFD+STZ treatment at the start of experiment, indicating induction of insulin resistance. On weekly observation, it was noted that all treatment groups gradually, week by week restored the HFD+STZ depleted QUICKI values indicating alleviation of insulin resistance. At the end of the study, all the three treatment groups restored to normal the HFD+STZ depleted QUICKI values indicating a reversal of insulin resistance.

The Matsuda index is calculated on the basis of BGL and insulin levels obtained during different time points in the OGTT. A Matsuda value of less than 4.3 indicates insulin resistance. The HFD+STZ treated group of animals exhibited a value of 2.644 which clearly indicates insulin resistance as against the Normal Control value of 4.47. All treatment groups exhibited significantly higher values than the HFD+STZ treatment group indicating a mitigation of insulin resistance. Herbal formulation1000 was comparable to Metformin treatment in reversing insulin resistance.

Herbal formulation of Example 4 (500 mg/kg) and Herbal formulation of Example 4 (1000 mg/kg) successfully attenuated the HFD+STZ elevated blood glucose and insulin levels. The Herbal formulations were comparable to Metformin 120 mg/kg in this regard. Herbal formulation of Example 4 (1000 mg/kg) was insignificantly better than Herbal formulation of Example 4 (500 mg/kg) in reducing the risen BGL and insulin levels.

The effect of Herbal formulation on HFD and STZ induced insulin resistance was evaluated by 3 methods. In all the 3 methods, Herbal formulations were comparable to metformin in ameliorating insulin resistance. Herbal formulation of Example 4 (1000 mg/kg) was insignificantly better than Herbal formulation of Example 4 (500 mg/kg) in alleviating insulin resistance.

1. A novel synergistic herbal formulation comprising;
a. 20% to 30% by w/w of Curcuma longa alcohol extract,
b. 5% to 10% by w/w of Curcuma longa water extract,
c. 1% to 10% by w/w of Turmerone oil,
d. 20% to 50% by w/w of Phylanthus emblica juice powder,
e. 20% to 50% by w/w of Phylanthus emblica extract,
f. 10% to 15% by w/w of Phosphatidyl choline

2. The novel synergistic herbal formulation as claimed in claim 1 wherein Curcuma longa alcohol extract and water extract used in said formulation is extracted from Curcuma Longa Rhizomes and is 90% to 95% Curcuminoids.

3. The novel synergistic herbal formulation as claimed in claim 1, wherein the Curcuma longa water extract contains 30% to 50% of polysaccharides and turmerone oil contains 50% to 70% α-turmerone.

4. The novel synergistic herbal formulation as claimed in claim 1, wherein Phylanthus emblica juice powder contains 0.2 to 2% Vitamin C and 4 to 5.5% Gallic acid.

5. The novel synergistic herbal formulation as claimed in claim 1, wherein Phylanthus emblica extract contains 30 to 45% of tannins and 10 to 12% Gallic acid.

6. The novel synergistic herbal formulation as claimed in claim 1, wherein Phosphatidyl choline used in said formulation is 40% to 90% non GMO soya lecithin or sunflower lecithin.

7. The novel synergistic herbal formulation as claimed in claim 1, wherein the formulation contains chromium-1 ppm to 3 ppm which is an insulin sensitizer.

8. The novel synergistic herbal formulation as claimed in claim 1, wherein the formulation processed as a powder to formulate into capsule, tablet, granules, sachets or liquid.

9. The novel synergistic herbal formulation as claimed in claim 1, wherein the formulation reduces insulin resistance and blood glucose levels in diabetic patients.

10. The novel synergistic herbal formulation as claimed in claim 1, wherein the said formulation is used in pre diabetic condition, diabetes mellitus, insulin resistant diabetes, Polycystic ovarian disorder (PCOS).

11. The novel synergistic herbal formulation as claimed in claim 1, wherein the dose of formulation in human is 500 mg per day to 2000 mg/day.
